(19) United States
(12) Patent Application Publication
(10) Pub. No.: US 2024/0342509 A1
(43) Pub. Date: Oct. 17, 2024

(54) DEVICE FOR APPLYING ULTRASOUND TO SUPERFICIAL TARGETS

(71) Applicants: Ross Duncan Harper, Calgary (CA); Dawn Annette Crocker, Calgary (CA)

(72) Inventors: Ross Duncan Harper, Calgary (CA); Dawn Annette Crocker, Calgary (CA); Laura Curiel, Calgary (CA); Seyed Mohammad Mohammadi Jahromi, Calgary (CA)

(73) Assignees: Ross Duncan Harper, Calgary, AB (CA); Dawn Annette Crocker, Calgary, AB (CA)

(21) Appl. No.: 18/631,128

(22) Filed: Apr. 10, 2024

Related U.S. Application Data
(60) Provisional application No. 63/495,460, filed on Apr. 11, 2023.

(30) Foreign Application Priority Data
Apr. 11, 2023 (CA) 3,195,640

(51) Int. Cl. A61N 7/00

(52) U.S. Cl. CPC A61N 7/00; A61N 2007/0034; A61N 2007/0078

(57) ABSTRACT

A device for stationary delivery of focused ultrasound therapy to a body surface treatment area of a patient includes a plurality of ultrasound emitters arranged in a flexible array, each emitter of the plurality of emitters having a polygonic geometry and a plurality of edges. At least one edge of each emitter is adjacent to and forms a narrow gap between the at least one edge and an edge of at least one other emitter of the plurality of emitters. Each emitter has an external face and an opposite internal face. The flexible array provides an area of uniform emitter coverage so that uniform ultrasonic waves are applied to the body surface treatment area of the patient when the device is placed onto, so as to conform to, a curvature of the body surface treatment area, and the external face of each emitter is adjacent to the treatment area.

### CROSS-REFERENCE TO RELATED APPLICATIONS

This application claims the benefit of U.S. Provisional Application No. 63/495,460 and Canadian Patent Application No. 3,195,640, both filed on Apr. 11, 2023, and both entitled “Device for Applying Ultrasound to Superficial Targets”, both of which are incorporated herein by reference.

### FIELD

The present disclosure relates to devices for ultrasonic treatment of superficial targets on a human patient. In particular, the present disclosure relates to devices for applying ultrasound waves to superficial targets located at the epidermis and dermis layers of the skin, for treatment of medical and/or cosmetic skin conditions.

### BACKGROUND

The application of ultrasonic waves to human and animal bodies is well known in the medical field. One common application is to use ultrasound transducers (also referred to herein as “ultrasound emitters”), attached to a probe or wand, for medical diagnostic purposes. The ultrasonic waves travel beneath the surface of the skin and reflect off of soft tissue structures within the body, and the reflected waves may be interpreted to view the soft tissue structures of interest. Ultrasound diagnostics are used, for example, to monitor pregnancies and to detect a wide number of medical conditions.

More recently, ultrasound has also been applied for treating structures located deep inside the body. For example, focused ultrasonic waves may be applied to shrink or eliminate tumors or other structures located deep within the body. Advantageously, such therapies may allow for the non-invasive treatment of some types of cancer, as the focused ultrasonic waves may destroy the targeted tissue without affecting the surrounding tissue. Such medical treatment devices are configured so that the waves emitted by each emitter, in an array of emitters, will converge at the deep tissue target located inside the patient's body.

Applicant is aware of a commercially available product that is an ultrasonic probe mounted to a wand, for treatment of cosmetic dermatological conditions. The wand includes one ultrasound emitter mounted to one end of the wand, and the wand is swept across the surface area of the patient's skin to apply the ultrasonic waves to the skin and thereby treat the skin condition. As the wand is swept across the targeted surface area during treatment, it may be difficult to achieve an even application of the ultrasound waves across the surface area being treated.

Additionally, Applicant is aware of United States patent publication no. 2019/0328354. This patent application describes a conformable, ultra-thin (eg: 240 nm) piezoelectric transducer array for ultrasonic imaging. The structure includes a plurality of piezoelectric transducer elements disposed between two layers of silicone elastomer. A first patterned bi-layer includes patterned electrodes interconnecting the transducer elements, bonded to the silicone elastomer with a polyimide layer. The array of piezoelectric transducer elements is interconnected by the patterned electrodes, which are serpentine structures that act as bridges between the piezoelectric islands in the array. The serpentine structure of the electrodes, and the elasticity of the silicone layers, allows the array to be stretched by up to 50% without breaking the electrical connections.

In the U.S. Pat. No. 8,611,189, an acoustic coupling device is disclosed. The device includes a membrane filled with fluid. The membrane includes a pouch, on one surface, for interference-fitting the transducer. The opposite surface of the membrane conforms to the body surface of a patient. The membrane also includes an inlet and outlet for liquid circulation to cool the transducers. The membrane may also include pores for weeping liquid, to further facilitate acoustic coupling of the transducer to the body surface. The control system can adjust flow rate of the circulating fluid if needed to further cool the area in contact with the transducer.

United States patent publication no. 2021/0370103 discloses a method and device for providing sonodynamic therapy, whereby a sensitizer drug accumulates in a tumor, and is activated when exposed to ultrasonic waves to destroy the tumor while leaving intact the surrounding healthy tissue, which may be applied for example to treating brain tumors. The device includes a skull-shaped frame supporting the plurality of transducers, and a cooling device. The cooling device is a membrane having channels for circulating cooling fluid. The cooling device is placed onto the patient's head, and then the skull-shaped frame with the transducers is placed over the membrane, so the membrane is positioned between the patient's scalp and the transducers.

United States patent publication no. US 2016/0136462 discloses a portable and wearable ultrasound device, comprising two or more transducers attached by electrical leads to a controller.

The transducers are attached to the body of a patient using adhesive patches, the adhesive patches including a gel cup that couples to the transducer. The gel inside the gel cup provides the acoustic coupling between the skin of the user and the transducer, and the device is designed for delivery of ultrasonic waves into deep tissue.

U.S. Pat. No. 8,414,494 discloses a thin-profile ultrasound therapy probe for implementing therapy in confined spaces. The thin profile is achieved by incorporating a cooling fluid channel around the periphery of the housing, which removes heat generated by the transducer without needing a bulky cooling balloon. Acoustic coupling accomplished by applying a gel or using a thin pillow or membrane filled with a coupling fluid.

### SUMMARY

In one aspect of the present disclosure, it is desirable to provide a device capable of supplying ultrasound waves to a superficial target on the human body, for the treatment of medical and/or cosmetic conditions affecting the epidermis and/or dermis layers of the skin. Examples of conditions affecting the epidermis or dermis layers of the skin may include, but are not limited to, eczema, seborrhea, psoriasis and acne, which conditions may be treated with mild thermal ultrasonic therapy. Such conditions may often affect a large area of the patient's body, rather than being isolated to a small patch or area of the patient. In another aspect, some skin cancers may be effectively treated using the devices disclosed herein, in particular where the ultrasound treatment is used as an adjunctive therapy, in combination with other, well-known treatments and therapies for skin cancer. Other conditions that may be treatable by application of ultrasound waves to superficial targets on the surface of the body, include: dandruff, toenail fungus, removal of unwanted hair, pattern baldness, wrinkles, burns and scar tissue removal.

To provide effective treatment of such conditions, it is preferable to use a device that is configured to supply an even, substantially uniform amount of ultrasound waves across the targeted area requiring treatment, wherein the device is stationary during the treatment. Although commercially available products, in the form of a wand or similar device, are capable of supplying ultrasound waves across an area of a patient's skin or body targeted for treatment, such devices must be manually moved across the targeted treatment area. It is therefore difficult to ensure the same amount of energy is supplied evenly across the targeted treatment area using a wand that is manually swept across the targeted area during treatment, because there may be variances in the amount of time the emitters of the device are adjacent each portion of the entire treatment area and the distance between the surface of the emitters and the patient's skin, depending on the speed and pattern of how the manual sweeping of the device is performed. Thus, configuring a device with an array of ultrasound emitters, with the array having a sufficient surface area to cover the area of the patient targeted for treatment, allows the device to remain stationary while the ultrasound waves are applied to the targeted area.

A further challenge in supplying ultrasound waves to treat a superficial target on the body of a patient, is that most areas of the body are contoured, and some surface areas of the body have complex, contoured profiles or geometries. Thus, in one aspect of the present disclosure, a flexible array of ultrasound emitters is capable of conforming to a contoured surface of the body when the flexible array is placed onto the targeted area for treatment. In some embodiments, a bespoke treatment device having a flexible array of ultrasound emitters is provided, the bespoke treatment device being configured to conform to a complex, contoured surface of a body part of an individual patient, which for example may include a device having a mask-like support structure for the array of ultrasound emitters for supplying ultrasound waves evenly across the surface of the patient's face. Configuring a device having a flexible support for the array of ultrasound emitters allows the emitters to be positioned against the patient's skin, eliminating air or gaps that may occur between the emitters and the skin of the patient, with a layer of transmitting fluid in-between the skin and the array of emitters for transmitting the ultrasound waves from the emitter into the skin of the patient.

In another aspect of the present disclosure, the ultrasound treatment device may be selectively configured to perform diagnostic ultrasound scanning or imaging. Such a device may be used, for example, to both supply ultrasound waves for treatment of a superficial target on the surface of the patient's body, and perform diagnostics on the targeted area to monitor the effect of the supplied ultrasound treatment on the superficial target. Conveniently, in some embodiments of a treatment device having both a treatment mode and a diagnostic mode, the device may be configured to switch between a treatment mode and a diagnostic mode, so that both ultrasound treatment and ultrasound imaging may be performed on the patient using the same device.

In one aspect of the present disclosure, a device for stationary delivery of focused ultrasound therapy to a body surface treatment area of a patient is provided. The device includes a plurality of ultrasound emitters arranged in a flexible array, each emitter of the plurality of emitters having a polygonic geometry and a plurality of edges, wherein at least one edge of each emitter is adjacent to and forms a narrow gap between the at least one edge and an edge of at least one other emitter of the plurality of emitters, each emitter having an external face and an opposite internal face. The flexible array provides an area of uniform emitter coverage so that uniform ultrasonic waves are applied to the body surface treatment area of the patient when the device is placed onto, so as to conform to, a curvature of the body surface treatment area.

In some embodiments, the edge of each emitter of the plurality of emitters may be bonded to the edge of an adjacent emitter of the plurality of emitters by an adhesive, the adhesive forming a flexible, inelastic hinge between each pair of adjacent emitters. The adhesive may be a conductor or transmitter of the ultrasonic waves emitted by the flexible array of emitters.

In some embodiments, the internal face of each emitter may be mounted to a flexible, inelastic substrate or support.

In some embodiments, the distance across each narrow gap between any two emitters of the said plurality of emitters is equal to or less than 0.5 mm, and each edge of the plurality of edges of each emitter is radiused to direct a portion of the ultrasonic waves into the narrow gap between adjacent emitters. In some embodiments, the polygonic geometry of each emitter of the plurality of emitters is triangular. In some embodiments, the plurality of triangular emitters forms a triangulated irregular network (TIN). In an aspect of the present disclosure, the body surface treatment area may be a human face, and the configuration of the TIN may be based on a contour map generated from a scan of the human face to be treated. For example, the scan of the human face may be a LIDAR scan.

In some embodiments, the polygonic geometry of each emitter of the plurality of emitters is quadrilateral. A distance across each said narrow gap between any two adjacent emitters of the said plurality of emitters may be greater than 0.5 mm, and a strip emitter may be positioned across, so as to cover, the narrow gap in between any two adjacent emitters of the plurality of emitters, and wherein the strip emitters thereby forming a part of the flexible array.

In some embodiments, the substrate or support includes a plurality of apertures, each aperture of the plurality of apertures positioned within the narrow gap in between two adjacent edges of two emitters of the plurality of emitters.

In some embodiments, a distance across each narrow gap between any two emitters of the plurality of emitters is greater than 0.5 mm, and each emitter has a flexible, inelastic membrane mounted to the emitter's external surface, the flexible membrane filled with an ultrasound conducting or transmitting liquid. Each flexible membrane has a total volume, and in some embodiments each flexible membrane is filled up to 85% of the total volume with the ultrasound conducting/transmitting liquid so as to allow the flexible membrane to conform to a curvature of the body surface treatment area when placed on the body surface treatment area. In some embodiments, each flexible membrane is sealed.

In another aspect, the internal and external faces of each emitter are planar, and preferably the internal face is parallel to the corresponding external face of each emitter. In some embodiments, the internal face of each emitter is planar and the external face of each emitter may be convex.

In some embodiments the flexible array further includes a plurality of heat sensors, the plurality of heat sensors embedded in the substrate/support so as to monitor the heat generated by each emitter of the flexible array.

### BRIEF DESCRIPTION OF THE FIGURES

FIG. 1 is a side profile view of an embodiment of an ultrasound emitter array in accordance with an embodiment of the present disclosure.

FIG. 2 is a top profile view of an embodiment of a triangular ultrasound emitter.

FIG. 3 is a sectional view of the triangular ultrasound emitter of FIG. 2, taken along line 3-3 of FIG. 2, and mounted to a membrane in an array of emitters in accordance with an embodiment of the present disclosure.

FIG. 4 is a side profile view of an ultrasound emitter array, wherein each emitter includes a fluid-filled membrane in accordance with an embodiment of the present disclosure.

FIG. 4A is a close up view of the ultrasound emitter array of FIG. 4.

FIG. 5 is an array of ultrasound emitters arranged in a triangular irregular network for conforming to the surface of a patient's face, in accordance with an embodiment of the present disclosure.

FIG. 6 is an embodiment of a composite emitter in accordance with the present disclosure.

FIG. 7A is a top profile view of another embodiment of an ultrasound emitter having radiused edges.

FIG. 7B is a sectional view of the ultrasound emitter shown in FIG. 7A, taken along the line 7B-7B of FIG. 7A.

FIG. 7C is a sectional view of an array of the ultrasound emitters of FIG. 7A, mounted to a flexible support.

### DETAILED DESCRIPTION

Although ultrasound is known for use in medical treatment and diagnostics, previous uses of ultrasound therapy have typically involved focused ultrasound treatment for directing ultrasound waves at soft tissue targets deep inside the body. It is also known to treat skin injuries, for example, by applying ultrasound waves to the injured area using an ultrasound emitter attached to a wand, the wand being a hand-held device that is typically swept across the targeted area to supply ultrasound waves to the targeted area. Similarly, ultrasound diagnostics have been typically used for medical imaging, detecting and monitoring medical conditions and diseases, again involving soft tissue structures located deep inside the body. Typically, the ultrasound imaging device includes an ultrasound emitter mounted to a wand that is manually swept back and forth across the surface of the body, proximate to the targeted area within the body and beneath the patient's skin.

Surprisingly, the Applicants have determined that ultrasound may also be used to treat medical and cosmetic conditions of the skin, which involves applying therapeutic ultrasound waves to superficial targets that are located on, or just beneath, the dermal layer of the human body. Examples of skin disorders or conditions that may be treated using ultrasound may include, but are not limited to: eczema, seborrhea, psoriasis, dandruff, toenail fungus, removal of unwanted hair, pattern baldness, wrinkles, burns, scar tissue removal, acne. Ultrasound, in some embodiments, may also be used to treat skin cancers and other diseases of the skin. Furthermore, there may be cosmetic applications for ultrasound involving the skin, including but not limited to non invasive body fat removal, and breast reduction or enlargement.

A challenge faced when applying ultrasound treatment to superficial targets on the human body include the need, in some cases, to apply a uniform, approximately equal amount of energy, in the form of ultrasound waves, to the targeted area of the patient's skin, which targeted area may typically be larger than the size of a single or multiple ultrasound emitters that may be typically mounted to a hand-held ultrasound wand device. Furthermore, the surfaces of the human body that may require treatment often include complex shapes or geometries, having contours, folds and ridges, which adds to the complexity of the challenge of supplying an even amount of energy produced by the ultrasound waves across the targeted area of the patient's skin. In the present disclosure, the applicant proposes different embodiments for devices which may be used for applying ultrasound waves to a superficial target on the human body.

In some embodiments, the devices disclosed herein may also include devices that are capable of imaging the targeted area, as well as supplying therapeutic ultrasound waves to the targeted area on the patient. Advantageously, in some embodiments, this may provide for both ultrasound treatment and ultrasound diagnostics of the targeted area on the patient's body, using the same device.

In some embodiments of the present disclosure, such as shown in FIG. 1, an ultrasound treatment device 1 includes a customized flexible or semi-flexible support 10 for supporting an array 20 of ultrasound emitters 22. Each emitter 22 in the array of ultrasound emitters 20 has a polygonal geometry and a plurality of edges. At least one edge of each emitter is adjacent to, and forms a narrow gap between, at least one edge of at least one other emitter of the plurality of the emitters that make up the array 20. Each emitter 22 includes an external planar face 22a, and then opposite internal face 22b, the internal face 22b of each emitter 22 being mounted to the flexible support 10. The external face 22a of each emitter 22 comes into contact with the patient's skin when the device is applied to the body surface treatment area of the patient. Electronic wiring 24, for connecting each emitter 22 to an electronic controller (not shown), may be partially incorporated into the flexible or semi-flexible support 10 of the device 1.

Optionally, in some embodiments the emitters 200 may include radiused edges 200c around the perimeter of the emitter 200. For example, as shown in FIGS. 7A to 7C, the emitter 200 includes substantially planar external and internal faces 220a, 220b, the external and internal faces 220a, 220b being substantially parallel to one another (as best viewed in FIG. 7B). However, because the edges 200c are radiused, this may allow for the plurality of emitters 200 in the array to be placed very close to each other, thereby creating a gap G of less than 0.5 mm, while still allowing for adjacent emitters 200 to move relative to one another, as the flexible support of the device moves to conform to the curvatures of a patient's treatment area. Advantageously, the radiused edges 200c may also direct ultrasound waves into the gap G between adjacent emitters 200, 200, thereby providing for uniform ultrasound wave coverage emitted from, and in between, the emitters 200 in the array of emitters. As best viewed in FIG. 7C, an array of emitters 200 mounted to a flexible support 210. The adjacent emitters 200 may move relative to one another, in direction X, as the device flexes and conforms to the surface of a patient's treatment area. Although the example of an emitter 200 having radiused edges 200c shown in FIG. 7A is in the shape of a triangle, it will be appreciated that emitters 200 having radiused edges 200c may have any polygonic geometry.

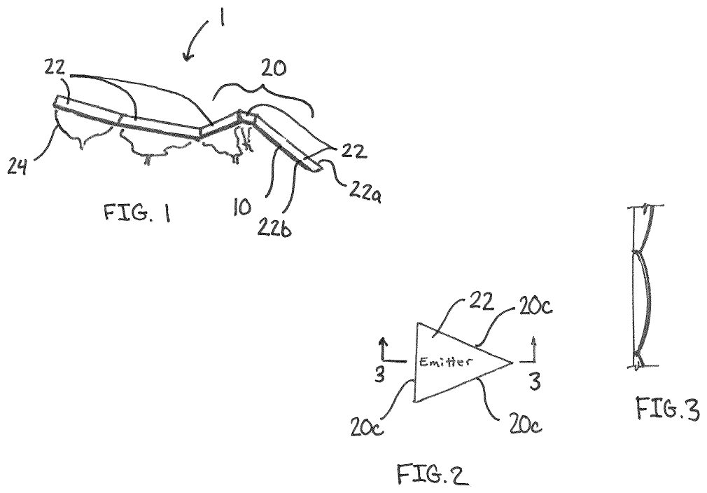

As may best be viewed in FIGS. 2 and 5, the polygonal geometry of each emitter 22 in the array of the emitters 20 may, in some embodiments, preferably be triangular. As best viewed in FIG. 5, a triangular geometry for each emitter 22 in the array of emitters 20 may be utilized to form a triangular irregular network (TIN). In some embodiments, a TIN may be utilized to construct an array of emitters 20 that conforms with the curvatures of a patient's body part or area, and the TIN may be customized to fit the body surface treatment area of a particular individual or patient.

The shape, curvature and profile of some body parts, for example an individual's face, is complex and varies between individuals. As such, in a preferred embodiment, a flexible or semi-flexible support may be constructed from a three-dimensional (3D) scan of a patient's face, as would be known to a person skilled in the art. Then, the array 20 of ultrasound emitters 22 may be constructed, for example, of a triangular irregular network to provide an area of uniform ultrasound emitter coverage across the entire curvature of the targeted body surface treatment area, including complex areas such as a human face. Advantageously, by using a TIN to configure the array 20 of emitters 22, the emitters are shaped, sized and then arranged to fit the exact size and shape of the patient's face, thereby providing a customized device for use in treating the patient. In some embodiments, computer modelling may be used to optimize the TIN, such that emitters having larger surface areas may be used to provide the required coverage, thereby reducing the overall number of emitters required to complete the TIN.

Additionally, it will be appreciated that the present disclosure is not limited to constructing a customized device fitted to a particular patient's face. For example, a flexible array may be constructed of an array 20 of emitters 22 such that the flexible support 10 may be sufficiently flexible and adaptable for placement on and use for treating the faces (or other body parts) of different patients, while sufficiently conforming to the curvature of that particular patient to thereby provide approximately uniform application of ultrasound waves to the treatment area of the patient. In particular, ultrasound treatment devices for use on less complex body parts may include an array of emitters 20 supported on a rectangular flexible membrane or a similar support 10. Devices for treating areas of the body, having simpler geometries and profiles, may not need to be customized in order to provide uniform ultrasound wave coverage to the targeted treatment area of the patient's body; for example, when the treatment area is on the back, abdomen, or a limb of the patient. However, for body surface treatment areas that are more complex, including for example the human face, a preferred embodiment includes a customized device that is preferably made to conform to a particular patient's face or body part. For larger treatment devices incorporating a large, flexible array of emitters, the flexible array may be supported on a large frame. The electronic wiring for controlling and driving the individual emitters may be incorporated into the frame, the frame making the treatment device easier to manipulate when positioning the treatment device onto a patient.

Again referring to FIG. 1, in some embodiments the flexible array 20 of ultrasound emitters 22 may not be mounted to a flexible support or membrane 10. Instead, the flexible array 20 may be constructed by using a flexible adhesive to attach the edge 20c of each emitter 22 to an adjacent edge 20c of an adjacent emitter 22. By adhering together the adjacent edges of each adjacent emitter of the plurality of emitters, with the cured adhesive forming a flexible hinge between the adjacent emitters, a flexible array of emitters 20 may advantageously be formed, without the bulk of a flexible membrane or other support 10.

Preferably, when treating a superficial target on the skin of a patient, the ultrasonic waves may be applied in a uniform manner such that each portion of the targeted treatment area on the patient's body receives an approximately equal amount of energy from the ultrasonic waves emitted by the array of emitters 20. As will be appreciated by a person skilled in the art, there are several challenges presented in providing substantially uniform energy from the ultrasonic waves across an area of the patient's skin that has been targeted for treatment. Preferably, ultrasonic waves are applied to the patient's skin such that the wavefront is approximately parallel to the patient's skin that is being treated. In some embodiments of the present disclosure, the external face of each emitter 22 in the array 20 is substantially flat or planar, such that when the emitter is adjacent to, and positioned against, the patient's skin, the ultrasonic waves emitted by the emitter present a wavefront that is substantially parallel to the surface of the patient's body. As the external face 22a of each emitter 22 is substantially planar, and a targeted treatment area of a patient's body is typically curved, providing a plurality of emitters arranged in a flexible array allows the treatment device 1 to substantially conform to the shape of the targeted treatment area. When the device 1 is positioned against the targeted treatment area such that the external face of each emitter in the array is positioned approximately parallel to, and adjacent to, the patient's skin, the relative flexing between adjacent emitters in the array allows the external surface of the entire device 1 to conform to the curvature of the patient's body.

An additional challenge in providing uniform emitter coverage across the targeted treatment area, is to address the small gaps that exist between the adjacent emitters in the array of emitters 20. The applicant has found that if an array of emitters 20 includes small gaps between the emitters, the resulting pattern of ultrasound waves emitted by the array 20 may include gaps between the wavefronts emitted by each individual emitter, which gaps in the wavefront pattern correspond to the gaps in between the adjacent emitters in the array. Therefore, the portions of the targeted treatment area that are adjacent to the gaps between the emitters may receive less or no energy from the ultrasound waves, as compared to the portions of the targeted treatment area that are directly adjacent to an emitter 22 when ultrasound therapy is being applied to the patient.

In some embodiments, the issue of gaps in the wave pattern emitted by the array of ultrasound emitters may be addressed by some combination of using an adhesive between the adjacent emitters in the array, which adhesive also serves as a transmitter of ultrasonic waves. In addition, or alternatively, a transmission fluid, such as a transmission gel or lotion capable of transmitting ultrasound waves, as would be known to a person skilled in the art, may be applied to the treatment area of the patient, and then the external face 22a of each emitter in the array of emitters 20 would be laid over the treatment area of the patient's body, with the layer of transmission fluid sandwiched in between the patient's skin and the treatment device 1.

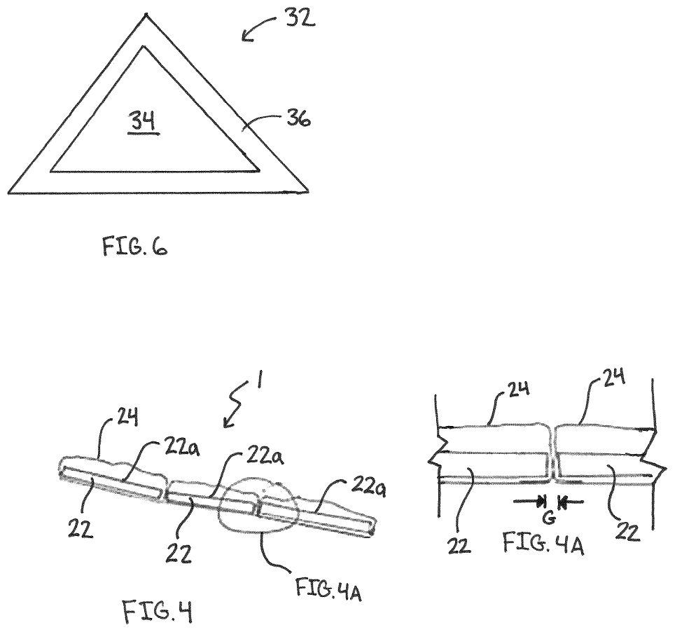

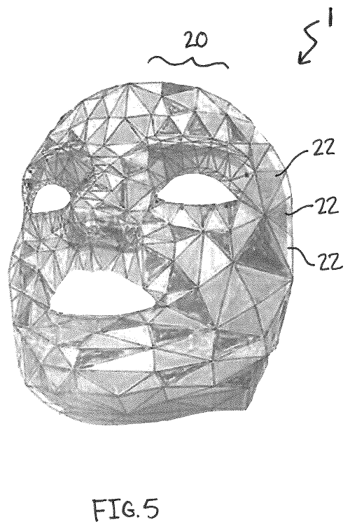

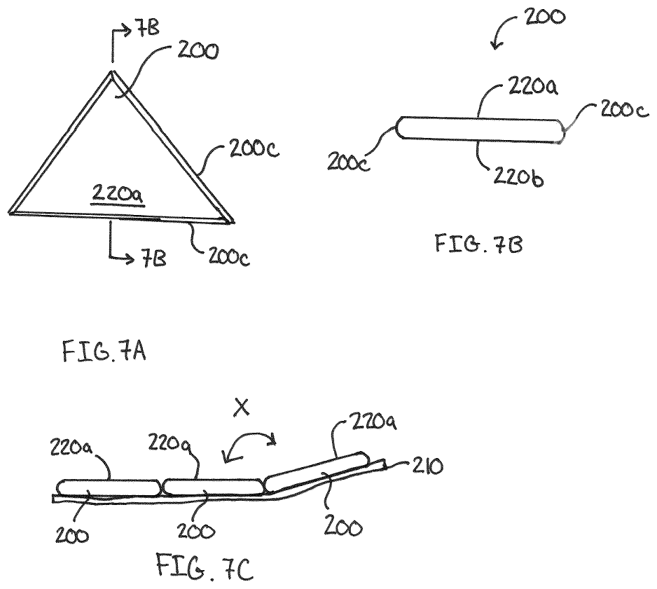

In other embodiments, such as shown in FIGS. 3 to 4A, each emitter 22 may include a flexible, inelastic membrane mounted to the ultrasound emitter 22. The flexible, in elastic membrane 24 may be filled with a transmission fluid for transmitting ultrasound waves. Advantageously, embodiments of the device including the flexible, inelastic membranes 24 may reduce or eliminate the need for applying the ultrasound transmission fluid to the patient's skin when using the device 1, which transmission fluid may be messy to handle, and may require re-application during treatment to ensure transmission of the ultrasound waves from the emitter to the patient's skin. Each membrane 24 may be filled with any suitable transmission fluid known to a person skilled in the art for transmitting ultrasound waves from the emitter to the patient's skin during treatment or imaging. As may best be viewed in the close-up of FIG. 4A, the flexible in elastic membranes 24 may, in some embodiments, be filled up to or less than 85% of the total volume up the flexible membrane 24 with transmission fluid. As viewed in FIG. 4, the membranes 24, being only partially filled with the conducting fluid, have malleable shapes or forms which allows the membrane to substantially conform to the curvatures of the patient's body when the device 1 is placed onto the patient. Because the emitters 22 in the array may be some distance apart, such that there is a gap G between adjacent emitters 22, the ultrasound waves that are emitted from the emitters 22 may have a gap in the wave pattern that corresponds to the gap G between the adjacent emitters 22, 22. However, does gap in the coverage of the uniform ultrasound waves emitted by the adjacent emitters 22, 22 in the emitter array 20 may be compensated for by the partially filled membranes 24 mounted to each of the emitters 22. Because the membranes are only partially filled with the transmitting liquid, the plurality of membranes 24, when placed on the skin of the patient, may contact each other, thereby forming a layer of touching, adjacent membranes that are filled with the transmission fluid. Such embodiments of the device 1 thereby provide a layer of transmission fluid, encased in a plurality of membranes, the layer sandwiched in between the array 20 of the emitters 22 and the surface of the patient, which then allows the ultrasound waves emitted by the adjacent emitters to be transmitted through the gap G between the adjacent emitters 22. As well, by providing a separate membrane mounted to each emitter, the overall shape of the layer of transmission fluid moves with, and conforms with, the flexible array of emitters. Thus, the applicant has found that the plurality of fluid filled membranes 24, mounted to the external face 22a of each emitter, may allow for uniform coverage of the treatment area with ultrasound waves transmitted through the plurality of membranes 24 from the plurality of the emitters 22.

In some embodiments, the gap G between adjacent emitters 22 may be covered by rectilinear, strip emitters (not shown). In such embodiments, the strip emitters may be positioned over each gap G in the array 20, such that the edges of the strip emitter overlap the edges of each adjacent emitter. The strip emitters may thereby cover the gap G between emitters 22, thereby providing complete coverage across the array 20 with a combination of overlapping emitters 22 and strip emitters. The strip emitters, for example positioned to be adjacent the external face 22a of the emitters 22, allows for the relative movement of the adjacent emitters in the array, while also covering the gap G between emitters 22. Preferably, the emitters 22 are arranged in the device so that the distance of the gap G is less than 0.5 mm; however, in some embodiments, the distance of the gap G may be equal to or greater than 0.5 mm.

For embodiments of the device comprising an array of emitters mounted to a flexible support or membrane, the support or membrane 10 may include a plurality of apertures. Each aperture is positioned within the gaps between adjacent emitters. Such apertures may provide pressure relief for breaking the suction between the membrane and the patient's skin when it is time to remove the device 1 from the patient after treatment has been completed. Otherwise, a flexible membrane or other support that does not include such apertures may suction onto the body, particularly when sweat is produced by the area of the body undergoing treatment, and the suction may be difficult to break when removing the device from the patient's body part, thereby causing discomfort to the patient when the device is removed. Thus, having one or more apertures passing through the flexible support or membrane may assist in breaking the suction between the device and the patient's body, thereby reducing or eliminating the discomfort that may otherwise occur during removal of the device. Additionally, the apertures may provide airflow to the targeted treatment area during use of the device, increasing the comfort of the patient and providing some cooling to the treatment area, as the application of ultrasound therapy often generates heat on the targeted treatment area. In some embodiments, the apertures may include short tubes which define the structure of the aperture and allow for sufficient airflow between the external and internal surfaces of the device.

In some embodiments, including treatment devices that are configured for treating body parts with fewer contours and generally less complicated shapes, the array of emitters may include other geometric shapes, including but not limited to rectilinear shapes. In some embodiments, the array of emitters may be supported on a flexible, inelastic support, such as a flexible membrane. In one embodiment, the device comprises and array of emitters that are rectilinear supported on a flexible membrane. Advantageously, such devices may not need to be customized for an individual patient and may therefore be used to treat a number of different body parts on a number of different patients, in particular when such body parts do not have complex geometries. For example, such devices may be useful for treating a patient's back, abdomen, arms or legs, particularly when the targeted area for treatment is relatively smooth and flat, or includes only minor, gradual curves. For treatment of such areas, a device that is similar to a blanket or patch may be suitable, as the blanket or patch may be simply placed over the area to be treated, and the emitters may be, for example, a plurality of rectilinear emitters sized so as to conform to the gradual curvature of the targeted body part. For example, not intended to be limiting, such an array of emitters may include each individual emitter being square in shape and having a length and a width of 1.5 centimeters. One example of a flexible, inelastic membrane may be manufactured of, or include, Polytetrafluoroethylene (PTFE), although it will be appreciated that other flexible, inelastic materials, suitable for supporting an array of ultrasound emitters as would be known to a person skilled in the art, may be used.

### Diagnostic and Treatment Modes

in some embodiments, the devices for treatment of a patient's body part by delivery of focused ultrasound therapy to a superficial target on the patient's skin may also incorporate diagnostic capabilities, whereby the device may be switched into a diagnostic mode for performing ultrasound imaging on the body part being treated. Advantageously, having a device that is capable of both supplying focused ultrasound therapy to a body part and also performing ultrasound imaging on the same body part, may allow for a doctor or other medical practitioner to both supply therapy and perform diagnostics on the patient's targeted treatment area using the same device.

In one aspect, the treatment mode of such a device requires continuous, even application of ultrasound waves to the treatment area; whereas, the imaging slash diagnostic mode of the device requires applying a pulsing ultrasound wave to the treatment area. Switching between the treatment and diagnostic modes of the device, in some embodiments, requires controlling the pulsing and the frequency of the ultrasound emitters, as imaging requires high frequency pulses whereas treatment requires low frequency, continuous ultrasound emission. Preferably, in some embodiments the device would remain stationary while imaging of the treatment area is being performed. The applicant has found that some materials for manufacturing the emitters may be suitable for driving the emitters in both a continuous mode (for treatment) and in a pulsed mode (for imaging). For example, a piezoelectric material that may be suitable for switching between continuous and pulsing modes is known as PZT DL 47, which the applicant has found responds well to both continuous and pulsing emission of ultrasound waves. Another example for dual driving, which may be suitable, includes a material known as PZT DL 20. Such materials are made of lead zirconate titanate (PZT).

Switching the device 1 between diagnostic and treatment modes may be accomplished through an electronic controller of the device. In one aspect of a dual driving embodiment of the device, the applicant has found therapeutic ultrasound may interfere with the imaging mode of the device, because the continuous driving of the ultrasound emitters during treatment may produce noise in the image when the device is switched to a pulsing mode for diagnostic purposes. Thus, the applicant proposes that in some embodiments, a timed excitation may be implemented for producing diagnostic images containing reduced noise, and therefore, clearer images may result. For example, the device may firstly be used in a diagnostic mode to obtain the images of the targeted treatment area, prior to switching the device into the treatment mode for the continuous application of therapeutic ultrasound to the treatment area. In other embodiments, the applicant proposes that the electronic circuitry may be designed to embed short pulses for obtaining imaging during the continuous therapy mode. In such embodiments, diagnostics may be performed at intervals during the ultrasound treatment session.

In a further embodiment of a device capable of switching between diagnostic and treatment modes, a dual emitter (otherwise referred to as a composite emitter) may be designed such that there are two or more portions of each emitter in the array of emitters. For example, as illustrated in FIG. 6, an emitter 32 may be comprised of a centre portion 34 which is driven by a first control circuit and designed for imaging and diagnostics, and a peripheral portion 36 of the emitter that is arranged peripherally around, and surrounds, the central portion 34 of the emitter 32, and is designed for continuous mode narrowband ultrasound generation, which supplies the continuous ultrasound waves for therapeutic application of ultrasound to the targeted treatment area. In this case, the second, peripheral portion would be driven by a separate circuit from the first, central portion, which would avoid having to switch the circuitry between the two different modes.

The applicant hypothesizes that producing a composite emitter, having a central portion and a separately controlled peripheral portion, may reduce the amount of noise in the images. The central portion of the emitter, for imaging, utilizes pulsing excitation, whereas the peripheral portion only utilizes continuous excitation, and therefore the continuous excitation of the peripheral portion would not, in such embodiments, affect the central portion which is used for imaging and diagnostics. Although the illustrated example of a composite emitter in FIG. 6 is a triangle, it will be appreciated that composite emitters may be designed to have any shape. In some embodiments, both the central portion and the peripheral portion of the composite emitter are manufactured of the same piezoelectric material. In other embodiments, the central and peripheral portions may be manufactured of different piezoelectric materials, wherein the material selected for the central portion of the composite emitter may be better suited for pulsing excitation, and the material selected for the peripheral portion of the composite emitter may be better suited for continuous mode narrowband ultrasound generation.

1. A device for stationary delivery of focused ultrasound therapy to a body surface treatment area of a patient, the device comprising:
a plurality of ultrasound emitters arranged in a flexible array, each emitter of the plurality of emitters having a polygonic geometry and a plurality of edges, wherein at least one edge of each emitter is adjacent to and forms a narrow gap between the at least one edge and an edge of at least one other emitter of the plurality of emitters, each emitter having an external face and an opposite internal face,
wherein the flexible array provides an area of uniform emitter coverage so that uniform ultrasonic waves are applied to the body surface treatment area of the patient when the device is placed onto, so as to conform to, a curvature of the body surface treatment area.

2. The device of claim 1, wherein each edge of each emitter of the plurality of emitters is bonded to the edge of an adjacent emitter of the plurality of emitters by an adhesive, the adhesive forming a flexible, inelastic hinge between each pair of adjacent emitters.

3. The device of claim 2, wherein the adhesive is a conductor of the ultrasonic waves emitted by the flexible array.

4. The device of claim 1, wherein the internal face of each emitter is mounted to a flexible, inelastic substrate.

5. The device of claim 1, wherein a distance across each said narrow gap between any two emitters of the said plurality of emitters is equal to or less than 0.5 mm, and each edge of the plurality of edges of each emitter is radiused to direct a portion of the ultrasonic waves into the narrow gap between adjacent emitters.

6. The device of claim 5, wherein the polygonic geometry of each emitter of the plurality of emitters is triangular.

7. The device of claim 6, wherein the plurality of triangular emitters forms a triangulated irregular network (TIN).

8. The device of claim 7, wherein the body surface treatment area is a face, and wherein the configuration of the TIN is based on a contour map generated from a scan of the face.

9. The device of claim 8, wherein the scan is a LIDAR scan.

10. The device of claim 5, wherein the polygonic geometry of each emitter of the plurality of emitters is quadrilateral.

11. The device of claim 4, wherein a distance across each said narrow gap between any two adjacent emitters of the said plurality of emitters is greater than 0.5 mm, and a strip emitter is positioned across, so as to cover, the narrow gap in between any two adjacent emitters of the plurality of emitters, and wherein the said strip emitters form a part of the flexible array.

12. The device of claim 4, wherein the substrate includes a plurality of apertures, each aperture of the plurality of apertures positioned within the narrow gap in between two adjacent edges of two emitters of the plurality of emitters.

13. The device of claim 1, wherein a distance across each said narrow gap between any two emitters of the said plurality of emitters is greater than 0.5 mm, and each emitter has a flexible, inelastic membrane mounted to the emitter's external surface, the flexible membrane filled with an ultrasound conducting liquid.

14. The device of claim 13, wherein each flexible membrane has a total volume, and wherein each flexible membrane is filled up to 85% of the total volume with the ultrasound conducting liquid so as to allow the flexible membrane to conform to a curvature of the body surface treatment area when placed on the body surface treatment area.

15. The device of claim 13, wherein each flexible membrane is sealed.

16. The device of claim 1, wherein the internal and external faces of each emitter are planar, and wherein the internal face is parallel to the corresponding external face of each emitter.

17. The device of claim 1, wherein the internal face of each emitter is planar and the external face of each emitter is convex.

18. The device of claim 4, wherein the flexible array further includes a plurality of heat sensors, the plurality of heat sensors embedded in the substrate so as to monitor the heat generated by each emitter of the flexible array.
